(19) United States
(12) Patent Application Publication
(10) Pub. No.: US 2015/0065972 A1
(43) Pub. Date: Mar. 5, 2015

(54) Male Urine Collection Device

(71) Applicant: William Edward Mayo, San Diego, CA (US)

(72) Inventor: William Edward Mayo, San Diego, CA (US)

(21) Appl. No.: 14/188,757

(22) Filed: Feb. 25, 2014

Related U.S. Application Data
(60) Provisional application No. 61/871,078, filed on Aug. 28, 2013.

(51) Int. Cl. A61M 1/00

(52) U.S. Cl. CPC A61M 1/0019
USPC 604/349

(57) ABSTRACT

A male urine collection device is provided. The present invention comprises a container having a removable lid, a urine collection member, and a tube having a first end connected to the urine storage container lid and a second end connected to the elongated urine collection member. In whole, the device provides a continuous channel between the urine collection member and the container. The container, lid, tube, and urine collection member are all removable from each other so that the device can be easily washed. The device provides a means for bedridden male individuals to urinate when there is no assistance currently available to them and to communicate the urine directly into a receptacle without spillage and without requiring the user to uplift themselves from the bed.

### CROSS REFERENCE TO RELATED APPLICATION

This application claims the benefit of U.S. Provisional Application No. 61/871,078 filed on Aug. 28, 2013. The above identified patent application is herein incorporated by reference in its entirety to provide continuity of disclosure.

### BACKGROUND OF THE INVENTION

1. Field of the Invention

The present invention relates to urine collection devices. More specifically, the present invention relates to urine collection devices for bedridden males.

Many individuals are afflicted with health problems that impair their mobility and restrict them to beds, such as spinal injuries and obesity. These individuals often require assistance to go to the bathroom because it may be impossible or dangerous for them to attempt to go to leave their bed on their own due to their mobility issues. However, assistance may not be available in certain circumstances, such as in the middle of the night, potentially resulting in these bedridden individuals soiling themselves, which is uncomfortable, embarrassing, and requires effort to clean.

Some devices attempt to address these issues by providing bedpans or bedpan-like devices that have elongated receptacles into which a male can urinate. However, these devices have open tops that leave the urine exposed to the air around the individual. The exposed urine can smell and if the device is knocked over or otherwise jostled, the liquid contained within the bedpan can spill therefrom, creating a mess. Other devices provide bags or other liquid holding devices that have an elongated receptacle extending therefrom into which a male can urinate. However, these devices can be difficult to use for bedridden individuals because they are not easy to urinate into unless the individual is standing or in a vertical position.

The present invention provides a male urine collection device that has a container with a removable lid, a urine collection member, and a flexible tube connecting the container and the urine collection member. The removable lid makes it easy to empty liquid from the container, while also preventing spills or other accidents that are common with devices having configurations where the stored liquid is exposed. The flexible tube allows the present invention to be used by bedridden individuals from a number of different positions, allowing individuals with severe mobility issues to use the present invention without moving a substantial amount. The urine collection member is ideally configured for males to urinate into without any spillage, further preventing any possible accidents. Finally, the entire device is constructed from reusable and washable material so that the device can be used repeatedly.

2. Description of the Prior Art

Devices have been disclosed in the prior art that relate to bedpans for bedridden individuals or devices specifically adapted to collect urine from males. These include devices that have been patented and published in patent application publications. These devices generally relate to urine containers having a tubular portion attached thereto. The following is a list of devices deemed most relevant to the present disclosure, which are herein described for the purposes of highlighting and differentiating the unique aspects of the present invention, and further highlighting the drawbacks existing in the prior art.

One such device is U.S. Pat. No. 1,642,255 to Lowenstein, which discloses a bedpan that is adapted to be used by bedridden individuals. The device comprises a bedpan having an inflexible outwardly protruding portion that may be urinated into without having to hold the device. The present invention also comprises a urine collecting apparatus having an outwardly extending portion; however the present invention's outwardly extending portion is a flexible tube that can be used by a bedridden individual without having to get out of bed.

Another such device is U.S. Pat. No. 5,778,461 to Attaway, which discloses a bedpan with an integral urinal. The integral urinal comprises a flexible tubular portion that can be extended from the bedpan. The present invention also includes a flexible tubular portion, but it further includes a lid over the urine container, reducing the chance that any urine will spill from the container if the container is jostled.

U.S. Published Patent Application Publication No. 2005/0177070 to Levinson discloses a urine collection device comprising a urine receptor having an open perimeter that tapers to an outlet aperture, an elongated tubular member connected to the outlet aperture, and a means for coupling a container to the tube member. The present invention differs in two key respects. First, the present invention is designed to be used only by male users, rather than both by male and female users as with Levinson. Second, the container is an integral component of the present invention and therefore the flexible tubular portion is intended to specifically couple with the provided container.

Another such device is U.S. Published Patent Application Publication No. 2008/0262451 to Broden, which discloses a device for collecting urine comprising a flexible bag that has a member for receiving fluid extending therefrom. The present invention also comprises a container for collecting urine and a member for collecting urine, but it further comprises a flexible tube connecting these two separate components.

U.S. Published Patent Application Publication No. 2010/0145288 to Mathews discloses a urinal that is designed to avoid spills, which utilizes a tubular portion that has a plurality of flexible sections separating a number of rigid sections. The tubular portion of the present invention is composed of flexible material all along its length and does not utilize alternating sections of flexible and rigid material. Therefore, the tubular portion of the present invention is much more adaptable and easier to use for bedridden male individuals.

A novel male urine collection apparatus is provided. The present invention comprises a urine collection container, a member adapted for males to urinate into from a seated or laying position, and a flexible tubular portion connecting the two other components. The present invention allows men who are bedridden or otherwise have poor mobility to relieve themselves without soiling themselves. The lid of the present invention keeps liquid securely stored within the container, without any risk of spilling. However, the lid is also removable, allowing users to easily dispose of liquid stored within the container and to easily clean the container after use. It substantially diverges in design elements from the prior art and consequently it is clear that there is a need in the art for an improvement to existing urine collection devices. In this regard the instant invention substantially fulfills these needs.

### SUMMARY OF THE INVENTION

In view of the foregoing disadvantages inherent in the known types of urine collection devices now present in the prior art, the present invention provides a new urine collection device wherein the same can be utilized for providing convenience for the user when the user is bedridden or has poor mobility and thus has great difficulty rising in the middle of the night to relieve himself.

It is therefore an object of the present invention to provide a new and improved male urine collection device that has all of the advantages of the prior art and none of the disadvantages.

It is another object of the present invention to provide a male urine collection device that may be used without having to get out of bed.

Another object of the present invention is to provide a male urine collection device that has a lid to prevent spills and keep the urine stored within the container secure.

Yet another object of the present invention is to provide a male urine collection device that has a separable components so that the device is easy to clean.

Yet another object of the present invention is to provide a male urine collection device that has a flexible tube connecting the storage portion and the collection portion, allowing the device to be used from a number of different positions.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the present invention.

FIG. 2 shows a perspective view of the container portion of the present invention, with a call out showing the connection between the lid and the container.

FIG. 3 shows a perspective view of the collection portion of the present invention.

FIG. 4 shows a perspective view of the present invention in use.

### DETAILED DESCRIPTION OF THE INVENTION

Reference is made herein to the attached drawings. Like reference numerals are used throughout the drawings to depict like or similar elements of the male urine collection device. For the purposes of presenting a brief and clear description of the present invention, the preferred embodiment will be discussed as used for allowing a bedridden male user to relieve himself from a laying down position. The figures are intended for representative purposes only and should not be considered to be limiting in any respect.

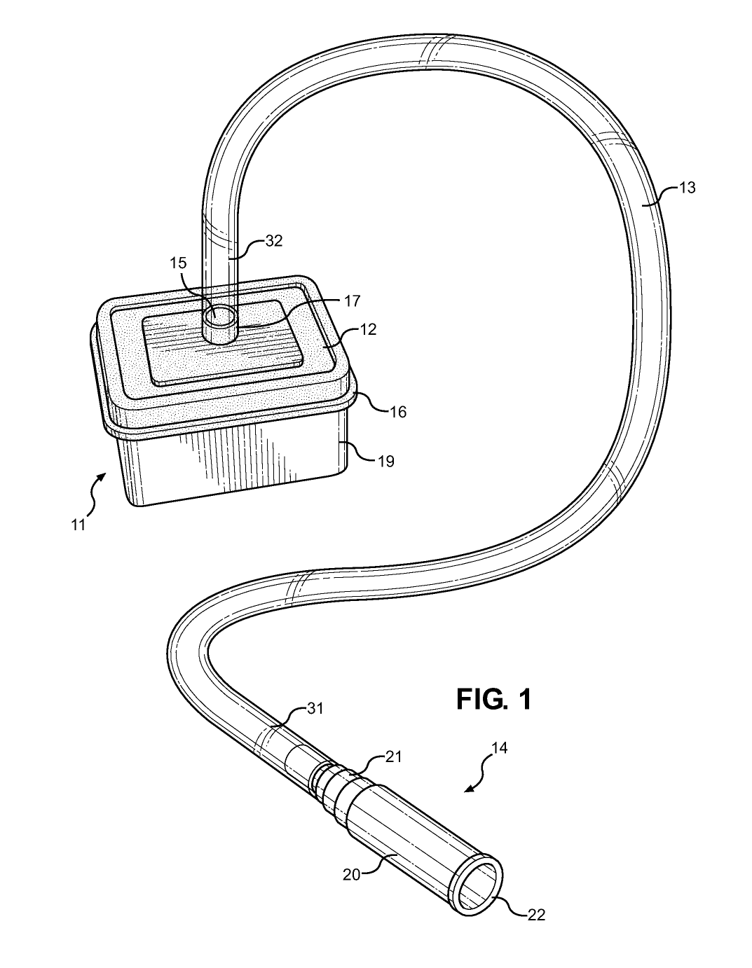

Referring now to FIG. 1, there is shown a perspective view of the present invention. The present male urine collection device comprises a container 11 having a removable lid 12, a urine collection member 14, and a tube 13 connecting the container 11 and the urine collection member 14. The present invention is adapted to allow a user who is bedridden or otherwise has mobility issues to urinate from his bed without having to get up or otherwise move, as the user could harm himself if he attempted to get up and go to the bathroom. The present invention is composed of reusable and readily-washable materials so the device can be cleaned out after each use and then used again as needed. The present invention is adapted for use by men suffering from all manner of afflictions affecting their mobility, including spinal injuries, obesity, and so on.

The container 11 of the present invention is composed of a base and a plurality of sidewalls 19 extending from the perimeter of the base, said sidewalls 19 having an upper edge that defines an open top. The container 11 is preferably composed of hard plastic, but no claim is made as to the material composition of the container 11, except that the material is preferably washable. As depicted, the container 11 has a rectangular base and rectangular sidewalls 19, but the container 11 of the present invention may be provided in all manner of shapes and sizes.

A lid 12 is provided for covering the open top of the container 11 and has a lid connection means 16 for removably affixing the lid 12 to the upper edge of the sidewalls 19. The lid 12 is preferably composed of soft plastic so the device can be easily removed from the container 11, but no claim is made as to the specific composition of the lid 12. The lid 12 has an aperture 15 and a first tube coupling means 17 surrounding and extending from the edges of the aperture 15. As depicted, the aperture 15 is circular and the first tube coupling means 17 comprises a cylindrical sidewall extending from the aperture 15, creating a channel. The aperture 15 is preferably disposed in the center of the lid 12, but no claim is made as to the precise location of the aperture 15 and it may be disposed at any point along the surface of the lid 12. The aperture 15 is provided to allow liquid to flow from the tube 13 into the container 11. The first tube coupling means 17 is provided to removably affix the lid 12 and the tube 13 together and to allow the free communication of fluid from the tube 13 through the lid 12 and into the container 11 without spillage or interruption. The first tube coupling means 17 generally comprises a cylindrical sidewall that has a diameter substantially equal to the tube interior diameter, or alternatively the tube exterior diameter, thereby allowing the first end 32 of the tube 13 to be securely, but removably, affixed onto the first tube coupling means 17. The first tube coupling means 17 has substantially the same cross-sectional shape as the tube 13 such that they may be fitted together. The tube 13 and the first tube coupling means 17 of the lid 12 may be affixed via the close tolerance between the two diameters thereof, or the connection may be facilitated by a hose clamp or similar clamping means (not shown) around the exterior of their connection area. It is submitted that this connection is well known in the art, wherein the present invention contemplates a secure connection that is also removable on demand to allow for separating and cleaning of each respective part (tube and lid).

The tube 13 comprises an elongated, generally cylindrical member having a hollow interior with a first end 32 and a second end 31. The second end 31 is adapted to attach to the urinal collection member 14 and the first end 32 is adapted to attach to the lid 12. The tube 13 provides an uninterrupted channel allowing liquid to flow from the urinal collection member 14 to the container 11. The tube 13 is composed of a flexible material, preferably soft plastic, which allows the present invention to be manipulated into a large number of different positions so that users can relieve themselves from any laying or sitting position. The present invention is designed for men who are bedridden or who otherwise have poor mobility, thus it needs to be highly adaptable to whatever position the user finds himself in so that a substantial amount of movement is not needed to utilize the device. Like the other components of the device, the tube 13 is preferably composed of reusable, washable material so that the present invention can be used for a number of times.

The urinal collection member 14 is roughly a substantially cylindrical or slightly conical funnel that comprises a hollow body 20 having a rim defining an inlet 22 at one end and a second tube coupling means 21 disposed at the opposite end. The urinal collection member 14 is designed such that a male user can urinate into the device without having to rise from a bed. As such, the second tube coupling means 21 is provided to direct the flow of the liquid into the tube 13 and the body 20 is provided to prevent urine from splashing out of the urinal collection member 14 and to give users an elongated member that is easy to urinate into. The second tube coupling means 21 preferably comprises a series of stepped members of decreasing diameter, which can be inserted within the tube 13 and then gradually expand the tube 13 as they are pushed in, generating a secure seal between the second tube coupling means 21 and the tube 13. It is submitted that this connection is well known in the art, wherein the present invention contemplates a secure connection that is also removable on demand to allow for separating and cleaning of each respective part (tube and urinal collection member). The urinal collection member 14 is preferably composed of hard plastic. Like the other components of the device, the urine collection member 14 is preferably composed of reusable, washable material so that the present invention can be used for a number of times.

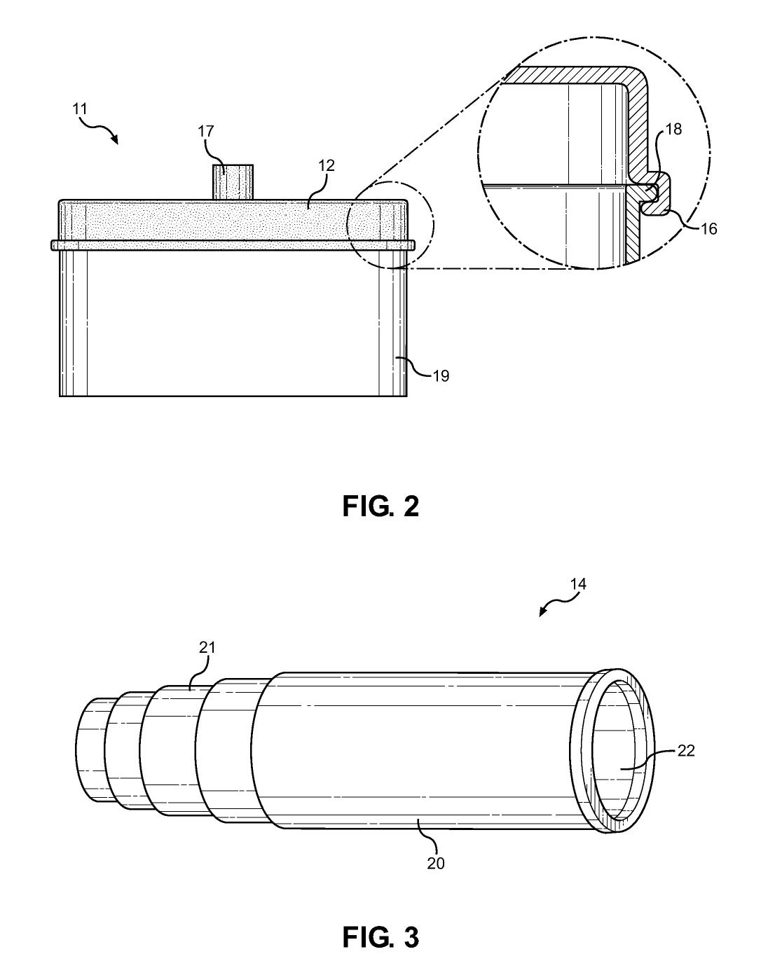

Referring now to FIG. 2, there is shown a perspective view of the container portion of the present invention, with a call out showing the connection between the container and the lid. The lid 12 is removably affixed on the upper edge 18 of the sidewalls 19 to cover the open top of the container 11. This prevents liquid stored within the container 11 from spilling therefrom when the container 11 is jostled. The lid 12 is also removable so that the liquid held within the container 11 can be easily disposed of and so that the present invention can be easily and thoroughly washed between uses. In the depicted embodiment, the lid connection means 16 comprises a notched, flexible flange portion on the lid 12 that engages with a complementary portion on the upper edge 18 of the container 11. No claim is made as to the precise mechanism for the lid connection means 16, as long as the lid 12 is removable. The lid connection means 16 may also comprises a plurality of latches that engage with complementary aligned notches on the upper edge of the sidewalls 19, hook and loop fastener material, or any other type of removable fastening means.

As depicted, the first tube coupling means 17 comprises a smooth, cylindrical sidewall extending from the central aperture that has a diameter substantially equal to that of the inner diameter of the tube 13. The tube 13 and the first tube coupling means 17 are affixed via the close tolerance between the two diameters thereof, or the connection may be facilitated by a hose clamp or similar clamping means (not shown) around the exterior of their connection area. Alternatively, the first tube coupling means 17 may comprise series of stepped members that gradually decreasing diameters, as seen for the urinal collection member of FIG. 3. However, no claim is made as to the precise structure of the first coupling means 17.

Referring now to FIG. 3, there is shown a perspective view of the urine collection member of the present invention. The urine collection member 14 generally comprises an elongated, cylindrical, or funnel-shaped member having a body 20 and a second tube coupling means 21. When all of the components of the present invention are connected together, the present invention provides a single, uninterrupted channel extending from the inlet 22 to the container 11, allowing liquid to flow from the former to the latter without spillage or interruption. The second tube coupling means 21 may comprise a series of stepped portions having an increasingly smaller diameter or may comprise a single smooth portion having a gradually decreasing diameter. In either case, the increasing diameter of the second tube coupling means 21 generates a secure connection between the tube 13 and the second tube coupling means 21 via the close communication between the inner diameter of the tube 13 and the diameter of the second tube coupling means 21.

The second tube coupling means 21 both allows liquid to flow therethrough into the tube 13 and secures the tube 13 and the urine collection member 14 together. The depicted embodiment of the second tube coupling means 21 comprises a series of stepped portions having gradually decreasing diameters. In use, the second tube coupling means 21 is placed within the interior of the second end 31 of the tube 13. The tube 13 and the urine collection member 14 are thereby held together by the close tolerance between the interior diameter of the second end 31 and the diameter of the second tube coupling means 21, or the connection may be facilitated by a hose clamp or similar clamping means (not shown) around the exterior of their connection area. However, no claim is made as to the precise structure of the second tube coupling means 21.

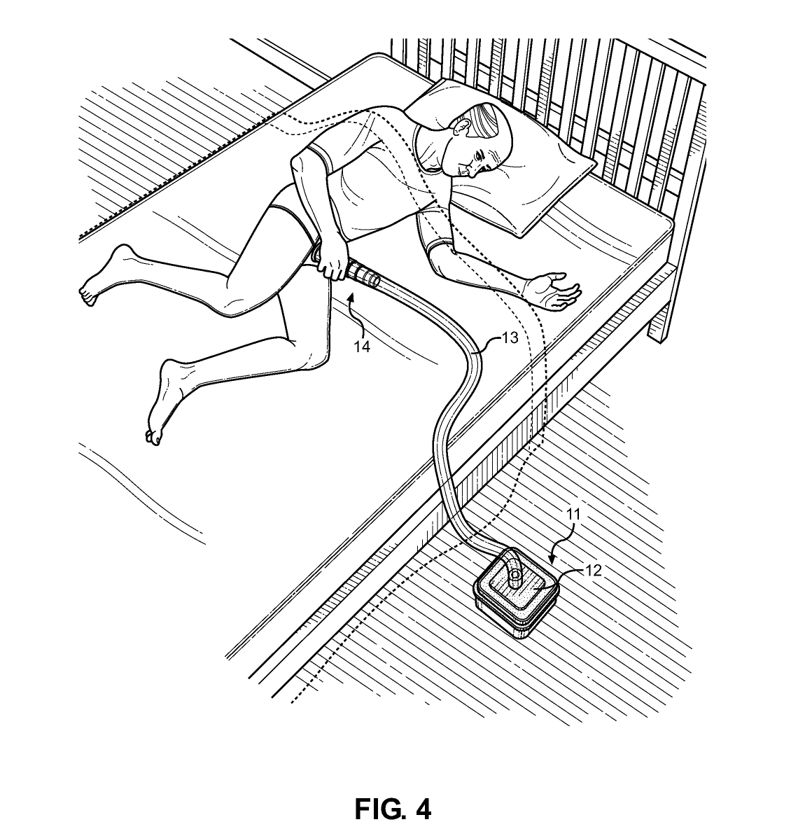

Referring now to FIG. 4, there is shown a perspective view of the present invention in use. The present invention is designed primarily for use by male individuals that are either bedridden or that have a substantial mobility impairment. It can be impossible or even dangerous for such individuals to attempt to rise from their bed, without assistance, to urinate. Therefore these individuals require a simple, reusable method for relieving themselves that is also clean and reduces the chance for any spills to occur.

Individuals utilize the present male urine collection device by positioning the urine collection member 14 as needed and then urinating into the urine collection member 14, through the inlet. The liquid automatically flows from the urine collection member 14, through the tube 13, to the container 11 where it is stored until it can be disposed of. The tube 13 is long and highly flexible, allowing it to be ideally positioned whether the user is lying on his side, sitting, or in any other position. Furthermore, the present invention is composed of washable material so that it can be readily reused.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A male urine collection device, comprising:
a container having a base and a plurality of sidewalls having an upper lip defining an open top;
a lid covering said open top and removably affixed along said upper lip of said sidewalls by a lid connection means;
said lid having a first tube coupling means extending therefrom;
said first tube coupling means having an aperture through said lid;
a tube composed of flexible material having a first end, a second end, an interior diameter, and an exterior diameter;
said first end of said tube removably connected to said first tube coupling means;
a urine collection member having an inlet, a body portion, and a second tube coupling means;
said second end of said tube removably connected to said second tube coupling means.

2) The male urine collection device of claim 1, wherein said container, lid, tube, and urine collection member are composed of washable plastic material.

3) The male urine collection device of claim 1, wherein said first tube coupling means comprises a cylindrical member having a diameter substantially equal to the interior diameter of said first end.

4) The male urine collection device of claim 1, wherein said second tube coupling means comprises a cylindrical member having a diameter substantially equal to the interior diameter of said second end.

5) The male urine collection device of claim 1, wherein said first tube coupling means comprises a stepped member having at least one diameter substantially equal to the interior diameter of the first end.

6) The male urine collection device of claim 1, wherein said second tube coupling means comprises a stepped member having at least one diameter substantially equal to the interior diameter of the second end.

7) The male urine collection device of claim 1, wherein said lid collection means comprises a notched, flexible flange extending around said lid that engages with a complementary portion on said upper lip.
